(19) United States
(12) Patent Application Publication
(10) Pub. No.: US 2008/0287772 A1
(43) Pub. Date: Nov. 20, 2008

(54) Motion Compensation in PET Reconstruction

(75) Inventors: Jerome Declerck, Oxford (GB); Kevin Scott Hakl, Oxford (GB)

Correspondence Address: CROWELL & MORING LLP;INTELLECTUAL PROPERTY GROUP, P.O. BOX 14300, WASHINGTON, DC, 20044-4300, US

(73) Assignee: Siemens Medical Solutions USA, Inc., Malvern, PA (US)

(21) Appl. No.: 12/121,245

(22) Filed: May 15, 2008

(30) Foreign Application Priority Data
May 18, 2007 (GB) 0709560.7
Sep. 26, 2007 (GB) 0718739.6

(51) Int. Cl. A61B 5/055; A61B 6/00

(52) U.S. Cl. 600/411; 382/131

(57) ABSTRACT

Method and apparatus motion compensation in Positron Emission Tomography (PET) imaging. PET and Magnetic Resonance Imaging (MRI) are performed simultaneously and the latter is used to determine motion of the subject during the acquisition process. The PET image data are then corrected according to the subject motion so determined.

Positron Emission Tomography (PET) is a nuclear medical imaging technique which provides a three-dimensional map of functional processes in the human or animal body.

A radioactive (positron emitting) tracer isotope is incorporated in a metabolically active molecule such as 18-F fluoro-2-deoxy-2-glucose (FDG, a sugar analogue) which is ingested in a subject. Other radiolabelled molecules which may be used in PET include 3′-deoxy-3′-[18-F]fluorothymidine (FLT) and 6-[(18)F]fluoro-L-DOPA (FDOPA). These molecules can be imaged by a scanner or camera which detects and records the gamma type radiation resulting from a collision between an emitted positron and an electron in the surrounding matter.

The radiation so produced is released as two photons travelling in near opposite directions. Hence, by detecting corresponding photons within a small co-incidence timing window (an event), a line of response (LOR) along which the origin of the radiation lies can be deduced.

The number of photons detected in coincidence between each pair of detectors (each pair forming a LOR) during a user-specified time interval is aggregated. Thus in its conventional form, PET is a counting modality. The measured counts in the LORs form an image known as a sinogram, that is effectively a projection, or Radon transform, of the original source distribution. Various techniques exist for back-projection of the sinogram to recover the estimated source distribution. These are mainly categorized into analytic methods based upon variations of the Filtered Back-Projection and iterative methods. The iterative methods are based on maximizing the log-likelihood of the data given the true source distribution and assume a strong statistical model based on Poisson statistics.

If the imaged subject moves during the image acquisition, the events will be recorded in different detector bins than the events before the subject moved. This causes an increase in image blur and reduced sensitivity to differences between hot and cold regions in the reconstructed images. In a worst case scenario additional hot lesions can be artificially generated by patient movement.

Patient motion has been shown in the literature to cause significant errors in the results of a PET exam in clinical brain studies amongst others. Head motion is particularly amenable to correction since the motion is generally rigid rotation and translation: compensating for these alone is sufficient for brain imaging if the motion is detected at the level of the skull.

In the past, this problem has been addressed by a number of approaches.

In one method, multiple dynamic frames are acquired and registered to each other or to a base-line post-reconstruction. This requires many dynamic frames to be reconstructed separately causing slow reconstruction times. In the case of rapid motion, very low statistics frames will be required which will cause difficulty with registration—often resulting in little in the way of correction.

In another method, events assigned to sinogram bins can be reassigned to the correct bins if the geometric transformations and times of motion occurrence are known a-priori. Alternatively, the transformations and times of motion can be incorporated in the reconstruction algorithm.

This approach has until now required the use of external camera monitoring equipment and small marks of some description attached to the subject head. This limits the technique to rigid transforms and external motion (such as brain imaging). Additionally the patient has to wear a device that contains the marks that the cameras detect. Finally, calibration of the camera equipment is non-trivial taking time and effort for each case.

An example commercial system employing this method is the Polaris system (Northern Digital, Inc, Waterloo, ON, Canada).

Another possible approach uses MR navigator echoes. A Navigator Echo is a quick MR pre-pulse sequence which measures the position of an organ before collecting raw data used for imaging. For instance, in thoracic imaging, navigator echoes trace the position of the diaphragm to monitor respiratory motion. Similarly, navigator echoes could be used to trace the scalp to monitor the motion of the head while imaging the brain. These monitoring signals are then used to create a gated image or to select the position or shape which is suitable to the clinician, or they can be used to correct for the motion itself.

The pre-pulse sequence acquires data over a narrow area perpendicular to the tracked structure. The contrast of the moving interface should obviously be high to permit easy automatic detection (diaphragm has high contrast between liver and lung, scalp has high contrast between head and air).

The advantage of this method is that it does not require additional equipment, but the drawback is that it takes scanning time to record a signal which cannot be used for imaging. Therefore, either the imaging time is increased or coincidence of the detected motion and imaging data is not possible to obtain.

Special Magnetic Resonance (MR) pulse sequences like BLADE which are designed to compensate for rigid patient motion by correcting acquired raw data directly in k-space are classically used for motion correction in MR imaging.

Combined MRI-PET systems are a recent development in the field of medical imaging. US 2005/0113667 discloses such a device, which allows for simultaneously performing MRI and PET scans on a subject.

According to the invention, a method of correcting a set of PET data for movement of a subject during PET data acquisition, said method comprises the steps set out in claim 1 attached hereto.

This invention addresses the problem of motion by monitoring the head position near continuously in order to provide correction factors that can be used either before or during PET reconstruction. The resulting motion compensated images will be more sensitive to contrasts between hot and cold regions and therefore will enable more accurate diagnosis to be made from PET scans. The advantage of this method over navigator echoes is that BLADE imaging enables the simultaneous acquisition of motion information and imaging data, which is essential for MR-PET imaging.

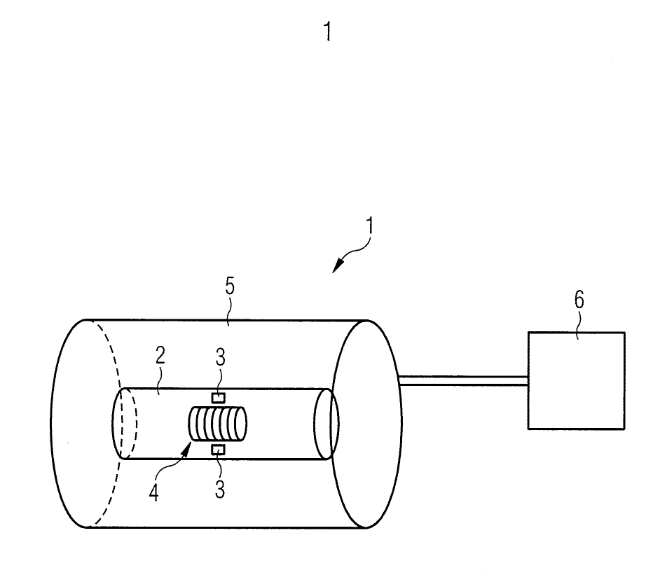

The invention will now be described, by way of example only, with reference to FIG. 1 which shows apparatus employed in a particular embodiment of the invention;

Referring to FIG. 1, apparatus of the invention, generally designated 1, includes a PET scanner 2 having a ring of scintillators 3 and a series of RF coils 4 located in the main magnet 5 of an MRI scanner (other components not shown).

Such a combination of PET and MRI scanning apparatus enables simultaneous and iso-volumic scanning of a volume of interest by PET and MRI. With such a device, a combination of a radioactive PET compound and a blood stream MRI contrast agent is injected simultaneously, and both PET and MR dynamic images are acquired.

The MR-PET apparatus are controlled by processor 6. Processor 6 could be any suitable computing apparatus, for example a desktop or laptop computer. Software applications associated with processor 6 allow user interaction to set parameters for a particular protocol and initiate scanning. Data acquisition and storage would also typically be controlled by processor 6.

Further applications provide for processing of data acquired during the MRI scan, as will be expanded on below, to determine subject movement during scanning and subsequent correction of PET data for such movement.

An MR acquisition pulse sequence is used, which is designed to track and compensate for motion in the raw data. The pulse sequence is used both to create the anatomical underlying image and to provide a list of transformation matrices (at small temporal regular sampling intervals) which models the rigid motion of the object. This pulse sequence is given as example: other sequences could be utilised so long as it is possible for each instance of the MR acquisition to get an update of a transformation matrix at a sufficiently fast rate. BLADE is designed to model rigid motion, but other motions (affine or even deformable) are possible with more complex pulse sequences.

For the reconstruction of the PET image, an iterative list-mode or sinogram-mode PET reconstruction algorithm can be employed, that uses the rotation matrix describing the object's transformation from its original position calculated from the list of transformations from the pulse sequence. The method by which the geometric correction factors are included in the reconstruction applies to iterative methods in which the probability that an event detected in a particular detector pair originated from a particular voxel can be written as P. In this case, the matrix P is factored into two components, W and G, where W represents the conventional sensitivities of detection, and G represents the geometric probability that an event in a LOR originated from a voxel. G incorporates the motion matrices derived from the transformation obtained from the BLADE sequence. In case of original misalignment between the PET and MR scans, an initial correction can be applied that is specific to each individual machine and verified using the BLADE scan.

Alternatively, a histogramming program that reassigns events to detector bins before reconstruction takes place can be used.

1. A method of correcting a set of PET data for movement of a subject during PET data acquisition, said method comprising the steps of:
simultaneously performing a PET scan and an MRI scan;
processing the results of the MRI scan to generate an MRI image and to determine motion of the subject during the scans and
correcting the data acquired from the PET scan for subject motion so detected.

2. A method according to claim 1 comprising the steps of:
acquiring MRI image data over a sequence of discrete time periods, T₀-Tₙ, during acquisition of the PET data, to produce a sequence of corresponding MRI datasets, D₀-Dₙ;
selecting an MRI dataset D₀, acquired over one of the time periods T₀ as a reference;
for each dataset, D₁, calculating a geometric transformation matrix Mᵢ representing the change in subject position between those represented by Dᵢ and D₀ respectively;
applying each matrix Mᵢ to the PET data acquired during Tᵢ to produce a set of corrected PET data and
reconstructing the corrected PET data to produce a set of PET images, corrected for movement of the subject during data acquisition.

3. A method according to claim 2, where the Geometric Transformation matrices are used, during reconstruction, to modify the Geometric value assigned to the probability of each event, associated with a particular pair of detector bins, occurring in a particular voxel.

4. A method according to claim 2, wherein the Geometric transformation matrices are used to reassign each event to a particular detector bin pair prior to image reconstruction.

5. Apparatus for correcting a set of PET data for movement of a subject during PET data acquisition comprising means for simultaneously performing a PET scan and an MRI scan on a subject and a processor arranged to:
receive data resulting from the PET and MRI scans;
process the data resulting from the MRI scan to determine movement of the subject during the scan and
correct the data resulting from the PET scan according to the movement so determined.
